(19) United States
(12) Patent Application Publication
(10) Pub. No.: US 2026/0232572 A1
(43) Pub. Date: Aug. 13, 2026

(54) INJECTABLE COMPOSITION, PHARMACEUTICAL FORMULATION INCLUDING THE SAME, AND METHOD FOR PREPARING THE COMPOSITION

(71) Applicants: ONCONIC THERAPEUTICS INC., Seoul (KR); JEIL PHARMACEUTICAL CO., LTD., Seoul (KR)

(72) Inventors: Aeri KIM, Gyeonggi-do (KR); Jungmin YUN, Gyeonggi-do (KR); John KIM, Seoul (KR)

(21) Appl. No.: 19/151,696

(22) PCT Filed: Jan. 31, 2024
(86) PCT No.: PCT/IB2024/050869

§ 371 Date: Jul. 29, 2025

(30) Foreign Application Priority Data
Feb. 1, 2023 (KR) 10-2023-0013947

(51) Int. Cl. A61K 9/00; A61K 9/08; A61K 9/19; A61K 31/437; A61K 47/26; A61K 47/40; A61K 47/42

(52) U.S. Cl. CPC A61K 9/0019; A61K 9/08; A61K 9/19; A61K 31/437; A61K 47/26; A61K 47/40; A61K 47/42

(57) ABSTRACT

The present invention relates to an injectable composition, a pharmaceutical formulation comprising the same, and a method for preparing the same The injectable composition according to the present invention comprises: azetidin-1-yl{8-[(2,6-dimethylbenzyl)amino]-2,3-dimethylimidazo[1,2-a]pyridin-6-yl}methanone citrate as active ingredient; and SBE-β-CD.

### TECHNICAL FIELD

The present invention relates to an injectable composition including an imidazo[1,2-a]pyridine compound or a salt thereof, a pharmaceutical formulation including the same, and a method for preparing the same.

### BACKGROUND

In general, a candidate substance (drug) needs to have not only desirable biological properties but also physical properties which enable a pharmaceutical use so that the it may be considered to be developed as a medication. However, in spite of having a very excellent activity, the drug often hardly applies to industrialization due to its low stability in a viewpoint of pharmaceutics.

In particular, among various kinds of formulations, an injectable formulation is injected into the body in a liquid state, and thus is required to have high solubility for injection solvent. Nevertheless, many drugs are poorly soluble or have low solubility or stability depending on pH conditions, and thus are difficult to be prepared into an injectable formulation. In order to solve these problems, an appropriate injection solvent needs to be selected along with a sufficient amount of additives such as a solubilizer, a stabilizer, and the like. However, a large amount of the additives may decrease stability, productivity, and the like, or may cause pain when injected into the body. Thus, it is very difficult to develop a formulation which may be used for injection along with excellent stability.

Meanwhile, an imidazo[1,2-a]pyridine compound or a pharmaceutically acceptable salt thereof is a medicinal raw material for preventing or treating gastrointestinal inflammatory diseases or gastric acid-related diseases such as peptic ulcer, gastroduodenal ulcer, gastritis, gastroesophageal reflux disease (GERD), non-erosive reflux disease (NERD), etc.

Accordingly, the present inventors have made extensive efforts to develop a very stable injectable formulation having excellent solubility and stability for the imidazo[1,2-a]pyridine compound or the pharmaceutically acceptable salt thereof, thereby completing the present invention.

### RELATED ART REFERENCES

### Patent Documents

(Patent Document 1) Korean Registered Patent Publication No. 10-1777971

### DETAILED DESCRIPTION OF THE INVENTION

### Technical Problem

One object of the present invention is to provide an injectable composition having excellent solubility and/or stability, including an imidazo[1,2-a]pyridine compound or a pharmaceutically acceptable salt thereof as an active ingredient.

Another object of the present invention is to provide a pharmaceutical formulation including the injectable composition.

Still another object of the present invention is to provide a kit including the pharmaceutical formulation.

Still another object of the present invention is to provide a method for preparing the injectable composition or the pharmaceutical formulation.

### Technical Solution

An injectable composition for one object of the present invention includes azetidin-1-yl{8-[(2,6-dimethylbenzyl)amino]-2,3-dimethylimidazo[1,2-a]pyridin-6-yl}methanone citrate as an active ingredient; and sulfobutylether-beta-cyclodextrin (hereinafter, SBE-β-CD).

In the present invention, azetidin-1-yl{8-[(2,6-dimethylbenzyl)amino]-2,3-dimethylimidazo[1,2-a]pyridin-6-yl}methanone is Compound 1 represented by chemical formula 1 below:

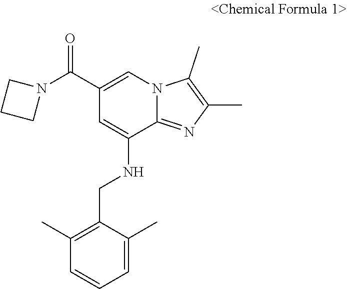 [SMILES: Cc1cccc(C)c1CNc1cc(C(=O)N2CCC2)cn2c(C)c(C)nc12]

The active ingredient in the present invention is citrate of Compound 1 represented by chemical formula 1, and hereinafter, “azetidin-1-yl{8-[(2,6-dimethylbenzyl)amino]-2,3-dimethylimidazo[1,2-a]pyridin-6-yl}methanone citrate” and “citrate of Compound 1” is referred to as the same thing as each other.

The citrate of Compound 1 may be represented by chemical formula 2 below.

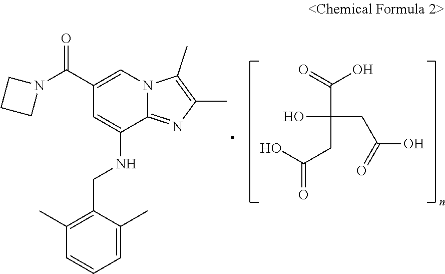

In above chemical formula 2, n may be 0.3 to 1.3, for example, 0.5 to 1.

The citrate of Compound 1, which is an active ingredient of the present invention shows far superior bioavailability compared to Compound 1, which is a free base, and is a drug showing a very excellent effect in preventing or treating gastrointestinal inflammatory diseases or gastric acid-related diseases. Specifically, when orally administered, citrate of Compound 1 reaches a highest blood concentration within a short time compared to Compound 1, which is a free base, Cmax is 11 times or more remarkably higher, and AUC may be also at least five times or more excellent compared thereto.

The citrate of Compound 1 shows very excellent solubility at below pH 3, but shows remarkably low solubility at a level of less than 1.5 mg/mL at pH 3 or higher, particularly at pH 3.3 or higher. However, the injectable composition according to the present invention may improve the solubility and stability of citrate of Compound 1 even at pH 3 or higher by using SBE-β-CD together with citrate of Compound 1

Even using 2-hydroxypropyl-β-cyclodextrin (HP-β-CD) and polyethylene glycol for citrate of Compound 1 may not increase the solubility of citrate of Compound 1 at pH 3 or higher, and polysorbate such as Tween 80 may not improve the stability of citrate of Compound 1. On the contrary, the injectable composition according to the present invention may include SBE-β-CD to improve the solubility and stability of citrate of Compound 1 at pH 3 or higher.

When a content of citrate of Compound 1 is less than 1 mg/mL, a large volume of injectable solution may need to be injected in order to exhibit a sufficient therapeutic effect, which may cause difficulty in administration.

In one embodiment, citrate of Compound 1 may be included in an amount of 1 to 10 wt % based on 100 wt % of the total solid content of the injectable composition. When the injectable composition is in a liquid phase, 100 wt % of the solid content may refer to a case in which the sum of the weights of the components excluding the injection solvent is 100%. When the injectable composition is in a solid phase, 100 wt % of the solid content may refer to a case in which a total weight of the injectable composition is 100%.

When an amount of citrate of Compound 1 is less than 1 wt % based on 100 wt % of the total solid content of the injectable composition, a large amount of injectable solution may need to be injected in order to exhibit a sufficient therapeutic effect, which may cause difficulty in administration. In addition, when citrate of Compound 1 is more than 10 wt % based on 100 wt % of the total solid content of the injectable composition, it may be difficult to sufficiently dissolve citrate of Compound 1, which may be easily precipitated.

In one embodiment, citrate of Compound 1 may be included in an amount of 1 mg or more and 30 mg or less, for example, 5 mg or more and 30 mg or less, per unit formulation of the injectable composition.

In the present invention, SBE-β-CD may be a dissolution aid for improving the solubility of citrate of Compound 1 at pH 3 or higher and/or a stabilizer for preventing citrate of Compound 1 from being precipitated.

In the present invention, SBE-β-CD may be a compound (sodium sulfobutylether-beta-cyclodextrin) represented by chemical formula 3 below having a form in which sodium is added to sulfobutylether-β-cyclodextrin.

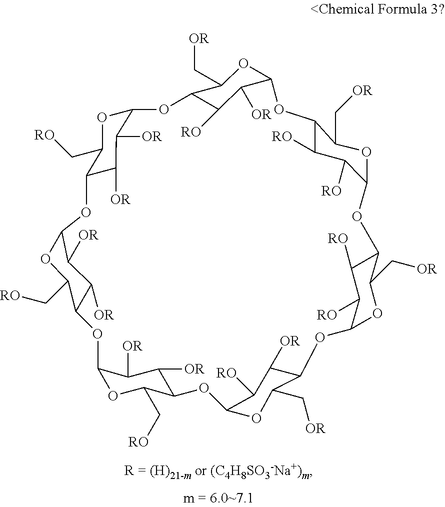

In one embodiment, in the injectable composition according to the present invention, a weight ratio of citrate of Compound 1 and SBE-β-CD may be 1:5 to 1:60 or 1:10 to 1:60.

In one embodiment, the injectable composition according to the present invention may include citrate of Compound 1; SBE-β-CD; and a pH adjuster. In this case, the pH adjuster may be used without limitation as long as it is a conventional pH adjuster to control pH in the preparation of an injectable composition such as sodium hydroxide.

In the present invention, the injectable composition including citrate of Compound 1 and SBE-β-CD may not need to include other additives in addition to the pH adjuster, but may further optionally include, without limitation, isotonic agents, buffers, osmotic agents, and the like commonly used in the art.

In one embodiment, the injectable composition according to the present invention may include citrate of Compound 1; SBE-β-CD; and albumin. In the present invention, when used with citrate of Compound 1 and SBE-β-CD, albumin may improve the solubility and stability of citrate of Compound 1 even at pH 3 or higher.

Albumin in the present invention may include recombinant albumin or albumin purified from human plasma.

In one embodiment, albumin may be in an amount of 1 mg or more and 60 mg or less per unit formulation of the injectable composition.

In one embodiment, albumin may be in an amount of 0.1 to 10 wt %, for example, 0.2 to 7 wt %, 0.25 to 7 wt %, 0.28 to 6.9 wt %, or 0.29 to 6.8 wt % based on 100 wt % of the total solid content of the injectable composition.

In one embodiment, an amount of albumin may be 105 mg to 1560 mg, specifically 106 mg to 1553 mg per 100 mL of the injectable composition, when a dosage form of the injectable composition is a liquid injectable formulation including an injection solvent.

In one embodiment, a weight ratio of SBE-β-CD and albumin may be 7:1 to 300:1.

In one embodiment, the injectable composition according to the present invention may include citrate of Compound 1; SBE-β-CD; and two or more selected from albumin, lysine, and arginine.

In one embodiment, the injectable composition according to the present invention may include citrate of Compound 1; SBE-β-CD; and lyophilizer.

Examples of the lyophilizer may include mannitol, trehalose, and the like, which may be used alone or in combination. The lyophilizer may further improve the stability of citrate of Compound 1 together with SBE-β-CD, and thus may improve the stability of the solid content or the freeze-dried product of the injectable composition.

In one embodiment, the injectable composition according to the present invention may include citrate of Compound 1; SBE-β-CD; and at least one or more of mannitol and trehalose.

In one embodiment, the injectable composition according to the present invention may include citrate of Compound 1; SBE-β-CD; albumin; and lyophilizer. The lyophilizer, together with SBE-β-CD and albumin, may further improve the stability of citrate of Compound 1.

In one embodiment, the injectable composition according to the present invention may include citrate of Compound 1; SBE-β-CD; albumin; and at least one or more of mannitol and trehalose.

In one embodiment, a weight ratio of citrate of Compound 1 and lyophilizer may be 1:5 to 1:10. For example, a weight ratio of citrate of Compound 1 and lyophilizer may be 1:7 to 1:8.

The injectable composition according to the present invention may be in a liquid phase or in a solid phase.

In one embodiment, the injectable composition according to the present invention may include citrate of Compound 1; SBE-β-CD; and an aqueous medium. In this case, the aqueous medium may be an injection solvent, and examples of the injection solvent may include, but are not particularly limited to, water for injection, physiological saline injectable solution, Ringer's solution, etc., and specifically, water for injection.

In one embodiment, the injectable composition according to the present invention may be colorless and transparent.

A pH of the injectable composition according to the present invention may be 3 or higher. An injectable formulation having pH of less than 3 may cause pain at the time of administration and increase a decomposition product from citrate of Compound 1, which is an active ingredient, and thus it may be preferable that at least the pH is 3 or more when considering safety and stability.

In one embodiment, the pH of the injectable composition according to the present invention may be 3 or more and 4.9 or less, may be 3 or more, 3.5 or more, 3.7 or more, 3.75 or more, 3.8 or more, 3.95 or more, or 4 or more, and may be 4.9 or less, 4.7 or less, or 4 or less. For example, the pH of the injectable composition according to the present invention may be 3 to 4.7, 3.5 to 4.9, 3.5 to 4.7, 3.5 to 4, 3.7 to 4, 3.75 to 4.7, 3.75 to 3.95, 4 to 4.7, 4 to 4.9, 3.75 to 4.9, or 3.75 to 4.

In one embodiment, the injectable composition according to the present invention may be a liquid formulation including citrate of Compound 1 and SBE-β-CD.

In one embodiment, the injectable composition according to the present invention may be a liquid formulation including citrate of Compound 1, SBE-β-CD and albumin.

In one embodiment, the injectable composition according to the present invention may be a colorless and transparent liquid formulation which includes citrate of Compound 1, SBE-β-CD and an aqueous medium, and has pH of 3 or more and 4.9 or less.

In one embodiment, the injectable composition according to the present invention may be a colorless and transparent liquid formation which includes citrate of Compound 1, SBE-β-CD, albumin, and an injection solvent, and has pH of 3 or more and 4.9 or less, for example, pH of 3.75 to 4.9.

In one embodiment, the injectable composition according to the present invention may be in a solid phase in the form of powder. The injectable composition in a liquid phase may be dried to be obtained in a powder form. Drying may be performed by a conventional drying process, for example, freeze drying, rotary evaporation drying, spray drying, fluid bed drying, or the like. In one embodiment, the injectable composition according to the present invention may be a freeze-dried product.

In one embodiment, the injectable composition according to the present invention may be a dried product including citrate of Compound 1 and SBE-β-CD.

In one embodiment, the injectable composition according to the present invention may be a dried product including citrate of Compound 1, SBE-β-CD, and lyophilizer.

In one embodiment, the injectable composition according to the present invention may be a dried product including citrate of Compound 1, SBE-β-CD and albumin.

In one embodiment, the injectable composition according to the present invention may be a dried product including citrate of Compound 1, SBE-β-CD, albumin and lyophilizer.

A pharmaceutical formulation for another object of the present invention may include citrate of Compound 1 and SBE-β-CD.

In one embodiment, the pharmaceutical formulation according to the present invention may be an injectable formulation including citrate of Compound 1 and SBE-β-CD. In this case, the injectable formulation may include an injection solvent.

The injectable formulation according to the present invention may have excellent solubility for active ingredients and have excellent stability against temperature and/or humidity.

In one embodiment, the pharmaceutical formulation according to the present invention may be an injectable formulation including citrate of Compound 1, SBE-β-CD and albumin. In this case, the injectable formulation may include an injection solvent.

In one embodiment, the pH of the injectable formulation including citrate of Compound 1 and SBE-β-CD may be 3 or higher and 4.9 or less.

In one embodiment, the pH of the injectable formulation including citrate of Compound 1 and SBE-β-CD may be 3.75 or higher and less than 4, or 4 or higher and 4.9 or less.

In one embodiment, the pharmaceutical formulation according to the present invention may be a freeze-dried product including citrate of Compound 1 and SBE-β-CD. In this case, the freeze-dried product may include mannitol and/or trehalose.

In one embodiment, the pharmaceutical formulation according to the present invention may be a freeze-dried product including citrate of Compound 1, SBE-β-CD and albumin. In this case, the freeze-dried product may include mannitol and/or trehalose.

The freeze-dried product according to the present invention may be stored for a long period of time by securing excellent stability against temperature and/or humidity, and may be easily formulated into an injectable formulation by being dissolving in the injection solvent as an aqueous medium. In this case, the pH of the obtained injectable formulation may be 3 or more and 4.9 or less, 3 or more and 4.7 or less, 3.75 or more and 4.9 or less, or 3.75 or more and 4.7 or less.

In one embodiment, a method for preparing an injectable composition according to the present invention may include mixing citrate of Compound 1; SBE-β-CD; and an aqueous medium. For example, the step may be performed by adding SBE-β-CD to an aqueous medium, and then mixing citrate of Compound 1 in an aqueous SBE-β-CD solution. The pH of the solution obtained by mixing citrate of Compound 1, SBE-β-CD, and the aqueous medium may be 3 to 4.9, specifically 3.5 to 4, or 3.75 to 3.95.

A liquid mixture including citrate of Compound 1 and SBE-β-CD may be an injectable formulation according to the present invention. The pH of the injectable formulation may be 3 to 4.9, specifically 3.5 to 4, or 3.75 to 3.95.

A process of freeze-drying the liquid mixture may be performed, and then the freeze-dried product according to the present invention may be prepared.

In one embodiment, a method for preparing an injectable composition according to the present invention may include mixing citrate of Compound 1, SBE-β-CD, albumin, and an aqueous medium. For example, the step may include: mixing citrate of Compound 1 and SBE-β-CD in an aqueous medium; and adding albumin to a mixed solution including citrate of Compound 1 and SBE-β-CD. The pH of the solution obtained by mixing citrate of Compound 1, SBE-β-CD, albumin and the aqueous medium may be 3 to 4.9, specifically 4 to 4.9, or 4 to 4.7.

The liquid mixture including citrate of Compound 1, SBE-β-CD and albumin, or the liquid mixture further including the pH adjuster therein may be the injectable formulation according to the present invention. The pH of the injectable formulation may be 3 to 4.7, specifically 4 to 4.9, or 4 to 4.7.

The preparation method may further include adding mannitol and/or trehalose to the liquid mixture.

The preparation method may further include drying the liquid mixture to which mannitol and/or trehalose is added. The drying may be performed through a freeze-drying process. In this case, the freeze-dried product according to the present invention may be prepared.

The freeze-dried product may be dissolved again in an aqueous medium such as an injection solvent, etc., to constitute a liquid injectable formulation. The freeze-dried product may be dissolved again to obtain a colorless and transparent property even when constituting a liquid injectable formulation. In this case, the pH of the liquid injectable formulation may be 3 to 4.9.

A kit according to the present invention may include: a freeze-dried product including azetidin-1-yl{8-[(2,6-dimethylbenzyl)amino]-2,3-dimethylimidazo[1,2-a]pyridin-6-yl}methanone citrate as active ingredient; and sulfobutylether-beta-cyclodextrin (SBE-β-CD); and an injection solvent.

The freeze-dried product in the kit is substantially the same as described above, and thus a repeated detailed description thereof will be omitted.

The injection solvent in the kit may be substantially the same as the aqueous medium described above. Thus, a repeated detailed description thereof will be omitted.

In the kit, the freeze-dried product and the injection solvent agent may be separately accommodated in each container to constitute one kit.
(1) The injectable composition according to the present invention includes: azetidin-1-yl{8-[(2,6-dimethylbenzyl)amino]-2,3-dimethylimidazo[1,2-a]pyridin-6-yl}methanone citrate as an active ingredient; and sulfobutylether-beta-cyclodextrin (SBE-β-CD).
(2) The injectable composition according to above (1) may further include albumin.
(3) The injectable composition according to above (1) or (2) may further include mannitol or trehalose as lyophilizer.
(4) The injectable composition according to any one of above (1) to (3) may further include albumin as a dissolution aid; and mannitol or trehalose as lyophilizer.
(5) The injectable composition according to any one of above (1) to (4) may be in a liquid phase with pH of 3 or more and 4.9 or less.
(6) The injectable composition according to any one of above (1) to (5) may be in a colorless and transparent liquid phase with pH of 3 to 3.95.
(7) The injectable composition according to any one of above (1) to (6) may be in a colorless and transparent liquid phase with pH of 4 to 4.9.
(8) The injectable composition according to any one of above (1) to (4) may be in a solid phase as a freeze-dried product.
(9) The pharmaceutical formulation according to the present invention includes: azetidin-1-yl{8-[(2,6-dimethylbenzyl)amino]-2,3-dimethylimidazo[1,2-a]pyridin-6-yl}methanone citrate as an active ingredient; and sulfobutylether-beta-cyclodextrin (SBE-β-CD).
(10) The pharmaceutical formulation according to above (9) may further include albumin.
(11) The pharmaceutical formulation according to above (9) or (10) may further include mannitol or trehalose as lyophilizer.
(12) The pharmaceutical formulation according to any one of above (9) to (11) may further include albumin; and mannitol or trehalose as lyophilizer.
(13) The pharmaceutical formulation according to any one of above (9) to (12) may be an injectable formulation or a freeze-dried product.
(14) The method for preparing the injectable composition according to the present invention includes: preparing an SBE-β-CD solution by mixing sulfobutylether-β-cyclodextrin (SBE-β-CD) and an aqueous medium; and mixing azetidin-1-yl{8-[(2,6-dimethylbenzyl)amino]-2,3-dimethylimidazo[1,2-a]pyridin-6-yl}methanone citrate in the SBE-β-CD solution.
(15) In the preparation method according to above (14), a solution having a colorless and transparent property may be obtained from the mixing of azetidin-1-yl{8-[(2,6-dimethylbenzyl)amino]-2,3-dimethylimidazo[1,2-a]pyridin-6-yl}methanone citrate.
(16) The preparation method according to above (14) or (15) may further include: mixing albumin, after mixing azetidin-1-yl{8-[(2,6-dimethylbenzyl)amino]-2,3-dimethylimidazo[1,2-a]pyridin-6-yl}methanone citrate.
(17) The preparation method according to above (16) may further include: drying a solution in which albumin is mixed.
(18) The preparation method according to above (16) or (17) may further include: adding mannitol or trehalose to a solution in which albumin is mixed; and drying the solution to which mannitol or trehalose is added.
(19) In the preparation method according to any one of above (14) to (18), the pH of the solution obtained from the mixing of azetidin-1-yl{8-[(2,6-dimethylbenzyl)amino]-2,3-dimethylimidazo[1,2-a]pyridin-6-yl}methanone citrate may be 3 to 4.9.
(20) In the preparation method according to any one of above (16) to (19), the pH of the solution in which albumin is mixed may be 4 to 4.7.
(21) In the preparation method according to any one of above (16) to (20), the pH of the solution in which albumin is mixed may be 4 to 4.9, and the preparation method may further include: drying the solution in which albumin is mixed.
(22) The injectable composition according to any one of above (1) to (8) may be a pharmaceutical composition for preventing or treating gastrointestinal inflammatory diseases or gastric acid-related diseases.
(23) The present invention provides a method for preventing or treating gastrointestinal inflammatory diseases or gastric acid-related diseases, the method comprising a therapeutically effective amount of the injectable composition according to any one of above (1) to (8).
(24) The present invention provides a use of the injectable composition according to any one of above (1) to (8) for preparing a medication for preventing or treating gastrointestinal inflammatory diseases or gastric acid-related diseases.
(25) The present invention provides a use of the injectable composition according to any one of above (1) to (8) for preventing or treating gastrointestinal inflammatory diseases or gastric acid-related diseases.

### Advantageous Effects

According to an injectable composition, a pharmaceutical formulation including the same, and a method for preparing the same according to the present invention, the injectable composition according to the present invention can secure excellent solubility and stability of azetidin-1-yl{8-[(2,6-dimethylbenzyl)amino]-2,3-dimethylimidazo[1,2-a]pyridin-6-yl}methanone citrate. It can be possible to provide an injectable formulation with excellent long-term storage stability and provide a freeze-dried product which is easily prepared as an injectable formulation, since there is no change in properties under high-temperature, high-humidity and/or long-term stress conditions in terms of stability, and azetidin-1-yl{8-[(2,6-dimethylbenzyl)amino]-2,3-dimethylimidazo[1,2-a]pyridin-6-yl}methanone citrate is not decomposed, but stably maintained.

Thus, the injectable composition including azetidin-1-yl{8-[(2,6-dimethylbenzyl)amino]-2,3-dimethylimidazo[1,2-a]pyridin-6-yl}methanone citrate with remarkably excellent bioavailability as an active ingredient can also have an excellent effect of preventing or treating gastrointestinal inflammatory diseases or gastric acid-related diseases.

### BRIEF DESCRIPTION OF THE DRAWINGS

FIG. 1 is a view showing pictures capable of confirming the properties of a freeze-dried product according to embodiments of the present invention.

### MODE FOR INVENTION

Hereinafter, the present invention will be described with reference to examples. The following examples are provided only for the purpose of illustrating the present invention, and thus the content of the present invention is not limited thereto.

### Preparation Example 1: Preparation of Citrate of Compound 1

Azetidin-1-yl{8-[(2,6-dimethylbenzyl)amino]-2,3-dimethylimidazo[1,2-a]pyridin-6-yl}methanone citrate was obtained according to a process below. Specifically, azetidin-1-yl{8-[(2,6-dimethylbenzyl)amino]-2,3-dimethylimidazo[1,2-a]pyridin-6-yl}methanone was obtained as described in Korean Registered Patent Publication No. 10-1777971. The results of NMR analysis on the obtained azetidin-1-yl{8-[(2,6-dimethylbenzyl)amino]-2,3-dimethylimidazo[1,2-a]pyridin-6-yl}methanone may be as follows.

¹H NMR (400 MHz, CDCl₃); δ 7.63 (d, J=1.2 Hz, 1H), 7.13 (dd, J=8.4, 6.8 Hz, 1H), 7.06-7.04 (m, 2H), 6.42 (d, J=1.2 Hz, 1H), 4.86-4.84 (m, 1H), 4.41-4.28 (m, 4H), 4.37 (d, J=4.4 Hz, 2H), 3.75-3.69 (m, 1H), 2.43-2.34 (m, 13H).

Subsequently, the obtained azetidin-1-yl{8-[(2,6-dimethylbenzyl)amino]-2,3-dimethylimidazo[1,2-a]pyridin-6-yl}methanone was mixed in an alcohol solvent (isopropyl alcohol, IPA) and stirred, and then dried under vacuum at about 30° C. to 35° C. to obtain a dried product, about 10 g of which was taken and stirred together with about 167 g of acetone. A solution obtained by dissolving citric acid (about 5 g) in acetone (about 33 g) was slowly added dropwise thereto for 60 minutes or more, and stirred at the same temperature for one hour. The resulting mixture was cooled to about 20° C. to 25° C. and further stirred for about one hour, after which the resulting solid was filtered, washed with acetone, and vacuum dried to obtain azetidin-1-yl{8-[(2,6-dimethylbenzyl)amino]-2,3-dimethylimidazo[1,2-a]pyridin-6-yl}methanone citrate. The results of NMR analysis on the obtained azetidin-1-yl{8-[(2,6-dimethylbenzyl)amino]-2,3-dimethylimidazo[1,2-a]pyridin-6-yl}methanone citrate may be as follows.

¹H NMR (400 MHz, MeOD); δ 7.90 (s, 1H), 7.06-7.15 (m, 3H), 6.77 (s, 1H), 4.50 (t, J=7.2 Hz, 2H), 4.45 (s, 2H), 4.24 (t, J=7.2 Hz, 2H), 2.80 (d, J=15.6 Hz, 2H), 2.70 (d, J=12.0, 2H), 2.39-2.44 (m, 11H), 2.35 (s, 3H).

### Examples 1 to 35: Preparation of Injectable Formulation

Azetidin-1-yl{8-[(2,6-dimethylbenzyl)amino]-2,3-dimethylimidazo[1,2-a]pyridin-6-yl}methanone citrate (citrate of Compound 1) obtained according to above Preparation Example 1, SBE-β-CD, and 10% albumin or mannitol were weighed in the amounts shown in Tables 1 to 4 below, mixed in a predetermined amount of water for injection, and completely dissolved by sufficiently stirring. The pH and properties of the obtained solution are also shown in Tables 1 to 4 below.

In the case of an injectable formulation including albumin in Examples of Tables 1 to 4 below, citrate of Compound 1 and SBE-β-CD, optionally mannitol, were mixed in water for injection and sufficiently stirred, and then 10% albumin was added in the content shown in Tables 1 to 4 and stirred.

TABLE 1

| | Example classification | | | | | | |
|---|---|---|---|---|---|---|---|
| Component | 1 | 2 | 3 | 4 | 5 | 6 | 7 |
| Citrate of Compound 1 (mg) | 5 | 5 | 5 | 5 | 5 | 5 | 5 |
| SBE-β-CD (mg) | 50 | 70 | 200 | 300 | 70 | 200 | 300 |
| 10% albumin (μL) | — | — | — | — | 50 (5) | 50 (5) | 50 (5) |
| (albumin amount, mg) | | | | | | | |
| Water for injection (μL) | 950 | 930 | 800 | 700 | 880 | 750 | 650 |
| pH | 3.75 | 3.8 | 3.8 | 3.95 | 4.5 | 4.5 | 4.5 |
| Property | Trans- | Trans- | Trans- | Trans- | Trans- | Trans- | Trans- |
| | parent | parent | parent | parent | parent | parent | parent |
| | solution | solution | solution | solution | solution | solution | solution |

TABLE 2

| | Example classification | | | | | | | | | | | |
|---|---|---|---|---|---|---|---|---|---|---|---|---|
| Component | 8 | 9 | 10 | 11 | 12 | 13 | 14 | 15 | 16 | 17 | 18 | 19 |
| Citrate of | 5 | 5 | 5 | 5 | 5 | 5 | 5 | 5 | 5 | 5 | 5 | 5 |
| Compound 1 (mg) | | | | | | | | | | | | |
| SBE-β-CD (mg) | 50 | 70 | 100 | 150 | 200 | 300 | 70 | 100 | 150 | 200 | 250 | 300 |
| 10% albumin (μL) | — | — | — | — | — | — | 10 (1) | 10 (1) | 10 (1) | 10 (1) | 10 (1) | 10 (1) |
| (albumin amount, mg) | | | | | | | | | | | | |
| Mannitol (mg) | 40 | 40 | 40 | 40 | 40 | 40 | 40 | 40 | 40 | 40 | 40 | 40 |
| Water for injection (μL) | 950 | 930 | 900 | 850 | 800 | 700 | 920 | 890 | 840 | 790 | 740 | 690 |
| pH | 3.75 | 3.83 | 3.82 | 3.79 | 3.89 | 3.91 | 4 | 4.01 | 4 | 4.04 | 4.06 | 4.1 |
| Property | Trans- | Trans- | Trans- | Trans- | Trans- | Trans- | Trans- | Trans- | Trans- | Trans- | Trans- | Trans- |
| | parent | parent | parent | parent | parent | parent | parent | parent | parent | parent | parent | parent |
| | solution | solution | solution | solution | solution | solution | solution | solution | solution | solution | solution | solution |

TABLE 3

| | Example classification | | | | | | | | | | | |
|---|---|---|---|---|---|---|---|---|---|---|---|---|
| Component | 20 | 21 | 22 | 23 | 24 | 25 | 26 | 27 | 28 | 29 | 30 | 31 |
| Citrate of | 5 | 5 | 5 | 5 | 5 | 5 | 5 | 5 | 5 | 5 | 5 | 5 |
| Compound 1 (mg) | | | | | | | | | | | | |
| SBE-β-CD (mg) | 70 | 100 | 150 | 200 | 250 | 300 | 100 | 150 | 250 | 70 | 100 | 150 |
| 10% albumin (μL) | 30 (3) | 30 (3) | 30 (3) | 30 (3) | 30 (3) | 30 (3) | 50 (5) | 50 (5) | 50 (5) | 100 (10) | 100 (10) | 100 (10) |
| (albumin amount, mg) | | | | | | | | | | | | |
| Mannitol (mg) | 40 | 40 | 40 | 40 | 40 | 40 | 40 | 40 | 40 | 40 | 40 | 40 |
| Water for injection (μL) | 900 | 870 | 820 | 770 | 720 | 670 | 850 | 800 | 700 | 830 | 800 | 750 |
| pH | 4.28 | 4.27 | 4.3 | 4.3 | 4.37 | 4.39 | 4.41 | 4.42 | 4.41 | 4.58 | 4.6 | 4.57 |
| Property | Trans- | Trans- | Trans- | Trans- | Trans- | Trans- | Trans- | Trans- | Trans- | Trans- | Trans- | Trans- |
| | parent | parent | parent | parent | parent | parent | parent | parent | parent | parent | parent | parent |
| | solution | solution | solution | solution | solution | solution | solution | solution | solution | solution | solution | solution |

TABLE 4

| | Example classification | | | |
|---|---|---|---|---|
| Component | 32 | 33 | 34 | 35 |
| Citrate of Compound 1 (mg) | 5 | 5 | 5 | 5 |
| SBE-β-CD (mg) | 200 | 250 | 300 | 300 |
| 10% albumin (μL) | 100 (10) | 100 (10) | 100 (10) | 50 (5) |
| (albumin amount, mg) | | | | |
| Mannitol (mg) | 40 | 40 | 40 | |
| Water for injection (μL) | 700 | 650 | 600 | 650 |
| pH | 4.58 | 4.65 | 4.64 | 4.5 |
| Property | Transparent | Transparent | Transparent | Transparent |
| | solution | solution | solution | solution |

Referring to above Tables 1 to 4, it can be confirmed that all the injectable formulations obtained according to Examples 1 to 35 were obtained as colorless and transparent solutions.

The citrate of Compound 1 may be used to obtain a transparent solution without SBE-β-CD under conditions of less than pH 3, but the solubility may rapidly decrease at pH 3 or higher, and citrate of Compound 1 may show excellent solubility at pH 3 to 4.7 due to SBE-β-CD. It can be confirmed that even if various known dissolution aids are used, the solubility of citrate of Compound 1 may not be improved in the range of pH 3 or higher, but solubility may be improved only due to SBE-β-CD by further including albumin and/or mannitol.

### Examples 36 to 52: Preparation of Freeze-Dried Product

Azetidin-1-yl{8-[(2,6-dimethylbenzyl)amino]-2,3-dimethylimidazo[1,2-a]pyridin-6-yl}methanone citrate (citrate of Compound 1) obtained according to above Preparation Example 1, SBE-β-CD and 10% albumin were weighed in the amounts shown in Table 5 below, mixed in a predetermined amount of water for injection, sufficiently stirred, and completely dissolved to obtain a colorless and transparent solution, or mannitol or trehalose was further added therein to obtain an injectable composition for preparing a freeze-dried product. In this case, the obtained solution was filtered through a 0.22 μm membrane filter, and the resulting solution was filled in a washed and sterilized sealable vial. After that, the vial was freeze-dried to prepare freeze-dried products according to Examples 36 to 43 of the present invention.

In the case of an injectable formulation including albumin in Examples of Table 5 below, citrate of Compound 1 and SBE-β-CD, optionally mannitol or trehalose, were mixed in water for injection and sufficiently stirred, and then 10% albumin was added in the content shown in Table 5 and stirred.

In addition, the liquid injectable compositions (Examples 2 to 5, 8 to 11, and 35) prepared according to above Tables 1 to 4 were freeze-dried by substantially the same process as described above, such as filtration, washing, etc., thereby preparing the freeze-dried products according to Examples 44 to 52 of the present invention.

TABLE 5

| | Example classification | | | | | | | |
|---|---|---|---|---|---|---|---|---|
| Component | 36 | 37 | 38 | 39 | 40 | 41 | 42 | 43 |
| Citrate of | 5 | 5 | 5 | 5 | 5 | 5 | 5 | 5 |
| Compound 1 (mg) | | | | | | | | |
| SBE-β-CD (mg) | 200 | 200 | 200 | 200 | 200 | 70 | 100 | 150 |
| 10% albumin (μL) | — | — | 150 (15) | 150 (15) | 150 (15) | 50 (5) | — | — |
| (albumin amount, mg) | | | | | | | | |
| Mannitol (mg) | 40 | — | — | 40 | — | 40 | — | — |
| Trehalose (mg) | — | 80 | — | — | 80 | — | — | — |
| Water for injection (μL) | 800 | 800 | 650 | 650 | 650 | 880 | 900 | 850 |
| pH | 3.8 | 3.8 | 4.9 | 4.9 | 4.9 | 4.5 | 3.8 | 3.8 |
| Property | White | White | White | White | White | White | White | White |
| | solid | solid | solid | solid | solid | solid | solid | solid |

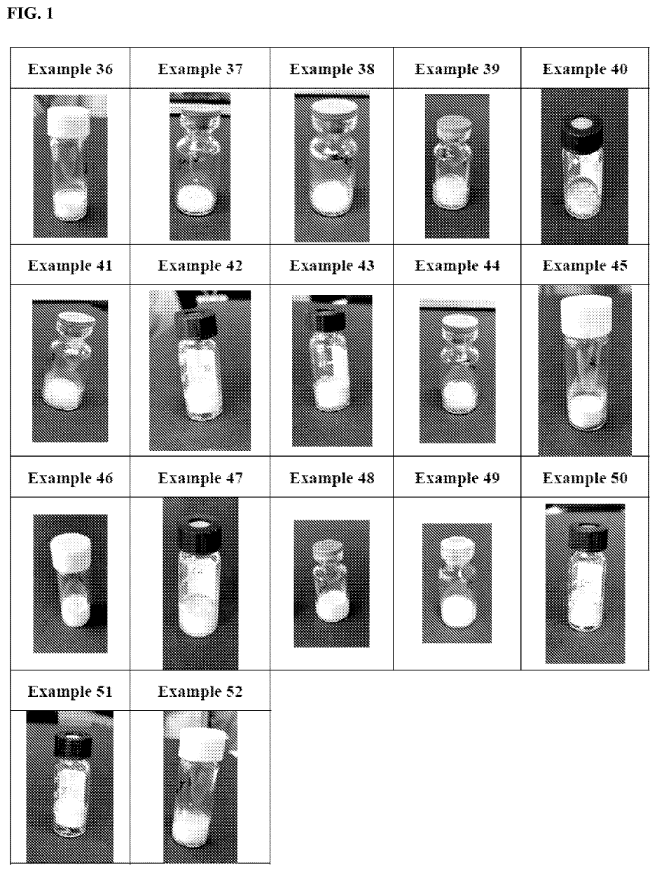

Pictures of FIG. 1 show properties of each freeze-dried product after being prepared according to Examples 36 to 52 of the present invention. Referring to FIG. 1, it can be confirmed that the freeze-dried products according to Examples 36 to 52 of the present invention were stably obtained as a white solid in a sufficient amount.

### Evaluation 1: Stability of Solution

For each injectable formulation according to an embodiment of the present invention, stability was confirmed for day 4 and day 7. Specifically, apparent changes and stability changes were confirmed using HPLC under conditions of 40° C., 25° C., and 4° C., respectively.

HPLC conditions may be as follows.

Column: C18 (4.6 mm×150 mm, 3 μm)

$$Mobile phase A = 0.1 \% TFA in water / B = acetonitrile$$

Mobile phase velocity: 1.0 ml/min

Detection: Ultraviolet absorbance photometer 254 nm

Mobile phase distribution over time

| Time (min) | A (%) | B (%) |
|---|---|---|
| 0 | 75 | 25 |
| 10 | 70 | 30 |
| 15 | 30 | 70 |
| 16 | 70 | 30 |
| 20 | 70 | 30 |

As a result, it could be confirmed that the injectable formulations according to Examples 3, 4, 12, 13, 17 to 19, 23 to 25, 28, and 7 of the present invention have no change in properties under refrigerated/room temperature/high temperature conditions even after at least five days.

In addition, the HPLC analysis results after seven days of the injectable formulations according to Examples 1, 2, 3, 4, 5, 6, and 35 of the present invention showed at least 99.5% or more, and thus it was confirmed that the injectable formulations are not actually decomposed but stably maintained.

When confirming storage stability under a condition of 4° C. considering that an injectable formulation is generally stored under a refrigerated condition, it can be confirmed that the injectable formulations according to the present invention illustrated in Tables 1 to 4 maintain initial colorless and transparent properties as they are for at least four days or longer under the refrigerated condition, and thus stability and storage stability under the refrigerated condition are excellent.

### Evaluation 2: Freeze-Dried Product Stability

For each of the freeze-dried products according to embodiments of the present invention, storage stability was evaluated for two weeks and four weeks under stress conditions (60° C., 80% RH). The results thereof are shown in Table 6 below.

TABLE 6

| | Example | | | | | | |
|---|---|---|---|---|---|---|---|
| | 36 | 37 | 38 | 39 | 41 | 44 | 49 |
| Properties after preparation | White solid | White solid | White solid | White solid | White solid | White solid | White solid |
| Properties after four weeks | White solid | White solid | White solid | White solid | White solid | White solid | White solid |
| Stability for two weeks, HPLC, % | 99.6 | 99.9 | 99.8 | 99.7 | 99.7 | 99.8 | 99.7 |
| Stability for four weeks, HPLC, % | 99.6 | 99.9 | 99.7 | 99.6 | 99.7 | 99.8 | 99.8 |

It was confirmed that the freeze-dried products according to the embodiments of the present invention may be stably obtained as a white solid, and are maintained as they are even after four weeks without discoloration or change in properties. In particular, it was confirmed that the HPLC analysis results show 99% or more, respectively, and thus the products are not actually decomposed, but stably maintained. Accordingly, it can be confirmed that the freeze-dried product according to the present invention has excellent stability.

In addition, the freeze-dried products stored at 60° C. and having properties and stability confirmed after four weeks were re-dissolved in 1 mL of distilled water after four weeks, and as a result, it was confirmed that a solution having a colorless and transparent property is obtained. In this case, it can be confirmed that the pH of the obtained solution is in the range of 3 to 4.9.

In addition, when the freeze-dried product of Example 41, which was stored at 4° C. and had its properties and stability confirmed after four weeks, was re-dissolved in 1 mL of distilled water, a solution having a colorless and transparent property was obtained.

The present invention has been described with reference to preferred exemplary embodiments herein, but it will be understood by those skilled in the art that the present invention may be variously changed and modified without departing from the spirit and field of the present invention, as described in the following scope of patent claims.

1. An injectable composition comprising:
azetidin-1-yl{8-[(2,6-dimethylbenzyl)amino]-2,3-dimethylimidazo[1,2-a]pyridin-6-yl}methanone citrate as active ingredient; and
sulfobutylether-β-cyclodextrin (SBE-β-CD).

2. The injectable composition of claim 1, further comprising albumin.

3. The injectable composition of claim 1, further comprising: mannitol or trehalose as lyophilizer.

4. The injectable composition of claim 1, further comprising:
albumin as a dissolution aid; and
mannitol or trehalose as lyophilizer.

5. The injectable composition of claim 1, wherein the injectable composition is in a liquid phase with pH of 3 or more and 4.9 or less.

6. The injectable composition of claim 1, wherein the injectable composition is in a colorless and transparent liquid phase with pH of 3 to 3.95.

7. The injectable composition of claim 2, wherein the injectable composition is in a colorless and transparent liquid phase with pH of 4 to 4.9.

8. The injectable composition of claim 1, wherein the injectable composition is in a solid phase as a freeze-dried product.

9. A pharmaceutical formulation comprising:
azetidin-1-yl{8-[(2,6-dimethylbenzyl)amino]-2,3-dimethylimidazo[1,2-a]pyridin-6-yl}methanone citrate as active ingredient; and
sulfobutylether-β-cyclodextrin (SBE-β-CD).

10. The pharmaceutical formulation of claim 9, further comprising: albumin.

11. The pharmaceutical formulation of claim 9, further comprising: mannitol or trehalose as lyophilizer.

12. The pharmaceutical formulation of claim 9, further comprising: albumin; and mannitol or trehalose as lyophilizer.

13. The pharmaceutical formulation of claim 9, wherein the pharmaceutical formulation is an injectable formulation or a freeze-dried product.

14. A method for preparing an injectable composition, the method comprising:
preparing an SBE-β-CD solution by mixing sulfobutylether-β-cyclodextrin (SBE-β-CD) and an aqueous medium; and
mixing azetidin-1-yl{8-[(2,6-dimethylbenzyl)amino]-2,3-dimethylimidazo[1,2-a]pyridin-6-yl}methanone citrate in the SBE-β-CD solution.

15. The method of claim 14, wherein a solution having a colorless and transparent property is obtained from the mixing of azetidin-1-yl{8-[(2,6-dimethylbenzyl)amino]-2,3-dimethylimidazo[1,2-a]pyridin-6-yl}methanone citrate.

16. The method of claim 14, further comprising: mixing azetidin-1-yl{8-[(2,6-dimethylbenzyl)amino]-2,3-dimethylimidazo[1,2-a]pyridin-6-yl}methanone citrate and then mixing albumin.

17. The method of claim 16, further comprising: drying a solution in which albumin is mixed.

18. The method of claim 14, further comprising:
adding mannitol or trehalose to a solution in which albumin is mixed; and
drying the solution to which mannitol or trehalose is added.

19. The method of claim 14, wherein the solution obtained from the mixing of azetidin-1-yl{8-[(2,6-dimethylbenzyl)amino]-2,3-dimethylimidazo[1,2-a]pyridin-6-yl}methanone citrate has pH of 3 to 4.9.

20. The method of claim 16, wherein the solution in which albumin is mixed has pH of 4 to 4.7.

21. The method of claim 16, wherein the solution in which albumin is mixed has pH of 4 to 4.9, the method further comprises:
drying the solution in which albumin is mixed.

22. (canceled)

23. A method for preventing or treating gastrointestinal inflammatory diseases or gastric acid-related diseases, the method comprising:
administering a therapeutically effective amount of the injectable composition according to claim 1 into a subject.

24.-25. (canceled)
